(19) United States
(12) Patent Application Publication
(10) Pub. No.: US 2009/0281630 A1
(43) Pub. Date: Nov. 12, 2009

(54) SHOULDER PROSTHESIS AND SET OF INSTRUMENTS FOR THE IMPLANTATION THEREOF

(75) Inventors: Philippe Delince, Brussels (BE); Etienne Lesur, Epinal Cedex (FR); Didier Oudet, Toijrs (FR); Francois Bruchou, Trappes (FR); Geraud Chaumeil, Albi (FR)

Correspondence Address: YOUNG & THOMPSON, 209 Madison Street, Suite 500, ALEXANDRIA, VA, 22314, US

(73) Assignees: TROIS S ORTHO, Lyon (FR); EVOLUTIS, Briennon (FR)

(21) Appl. No.: 12/306,865

(22) PCT Filed: Jun. 13, 2007
(86) PCT No.: PCT/FR07/00978

§ 371 Date: Jul. 27, 2009

(30) Foreign Application Priority Data
Jun. 28, 2006 (FR) 0605813

(51) Int. Cl. A61F 2/40

(52) U.S. Cl. 623/19.13; 623/19.11

(57) ABSTRACT

This shoulder prosthesis, comprising a glenoid element (6) intended to be attached to the scapula.

According to the invention,

this glenoid element (6) comprises a distal anchoring tail (20) and a proximal flange (21), the distal anchoring tail (20) having a substantially “I-shaped” cross-section, i.e. with a central core and two side walls substantially perpendicular to this central core, and with a helicoidally twisted shape, and the proximal flange (21) being perforated with at least one hole (30); and

the prosthesis comprises at least one screw (5) intended to be engaged into the hole (30) of said flange (21) and inserted into the bone.

### BACKGROUND OF THE INVENTION

The present invention relates to a shoulder prosthesis and to a set of instruments for implanting this prosthesis.

A shoulder prosthesis conventionally comprises a medullary rod intended to be introduced into the medullary channel of the humerus and a glenoid element intended to be anchored to the socket of the scapula, this medullary rod and this glenoid element including jointed surfaces allowing them to be mobile. These jointed surfaces may be formed by a convex rounded head mounted on the rod and a glenoid element attached to the scapula or, in a so-called “inverse” prosthesis, by a glenoid element mounted on the humeral rod and a convex rounded head attached to the scapula.

Documents FR 2 704 747, U.S. Pat. No. 5,030,219, FR 2 674 124, WO 01/47442 and EP 0 581 667 illustrate various shoulder prostheses

Upon implantation of a shoulder prosthesis, the placement of the glenoid element may be relatively delicate to achieve, and perfect attachment of this element is an essential condition for proper operation, and therefore for the durability of the prosthesis.

### SUMMARY OF THE INVENTION

The existing prostheses do not give complete satisfaction from this point of view, and an essential goal of the present invention is to provide a shoulder prosthesis, the glenoid element of which may be perfectly attached to this scapula, and this is relatively easy and rapid to apply.

According to the invention,
the glenoid element comprises a distal anchoring tail and a proximal flange, the distal anchoring tail having a substantially “I-shaped” cross-section, i.e. with a central core and two side walls substantially perpendicular to this central core, and with a helicoidally twisted shape, and the proximal flange being perforated with at least one hole; and
the prosthesis comprises at least one screw intended to be engaged into the hole of said flange and inserted into the bone.

The glenoid element is thus set into place in the scapula by helicoidal impaction in a perforation with a corresponding section made beforehand in the scapula by means of a suitable instrument, until the flange comes into contact with the bone, and at least one screw is then placed through the flange and then into the bone, in order to block the pivoting of this glenoid element.

This placement by helicoidal impaction, combined with the “I-shaped” section of the distal anchoring tail, provides perfect attachment of the glenoid element, with a support in the bone with a large surface area.

The set of instruments according to the invention comprises a perforating instrument, provided with a portion for perforating and hollowing out the bone of the socket of the scapula having a same shape and same dimensions as those of the distal anchoring tail of the glenoid element, or slightly smaller dimensions.

The distal helicoidal anchoring tail may comprise an osteo-inductive coating, notably a porous coating of calcium hydroxyapatite.

Advantageously, the distal end of the distal anchoring tail is arranged in the same way as the cutting end of a drilling bit, i.e. it has distal faces tilted on the proximal side from the axis of the distal anchoring tail and beveled according to inverse relief angles.

The distal anchoring tail may thus be more or less self-tapping; it may consequently be introduced into a bone perforation of adjusted dimensions, or slightly smaller than its own, which ensures perfect immobilization relatively to the bone.

According to one possibility, the distal anchoring tail comprises an axial bore allowing it to be engaged onto a guiding pin belonging to the set of instruments according to the invention; the perforating instrument comprises also in this case an axial bore allowing it to be engaged on this pin.

This perforating instrument may thus be accurately guided by this pin upon perforating the bone and the glenoid element may then in turn be accurately guided by this same pin upon its placement.

The proximal flange advantageously has a distal face with a convex rounded shape. This face allows the flange to come into contact with the bone over a large surface area.

This face may also comprise an osteo-inductive coating.

At least one of the holes of the flange may be delimited by a conical wall or a hollow sphere portion, allowing the screw to be implanted according to a plurality of possible angular positions relatively to the glenoid element.

Latitude in the positioning of the screw is thereby created, allowing the implantation of this screw under the best conditions.

The flange preferably comprises several holes, notably four holes, so that the positioning of one or more screws may be chosen.

The glenoid element also comprises mounting means on it for an element forming the jointed surface. These mounting means may notably comprise a low-pitched frustro-conical stud, onto which an element forming the jointed surface may be engaged, this element comprising a corresponding low-pitched frustro-conical cavity.

This stud may comprise an axial tapped bore with which the mounting of the element forming the jointed surface may be ensured by means of a screw. Such a screw is notably useful when the element forming the jointed surface comprises a ceramic head. The hole of this screw may optionally be used for ablating the jointed surface.

The set of instruments according to the invention may also comprise an instrument for locating the bone portion to be perforated, having a flattened circular head in which an aperture is made of the same profile as that of the “I-shaped” cross-section of the medullary anchoring tail.

### BRIEF DESCRIPTION OF THE DRAWINGS

The invention will be better understood, and other features and advantages thereof will become apparent, with reference to the appended schematic drawing, this drawing illustrating as a non-limiting example, a preferred embodiment of the shoulder prosthesis to which it relates.

FIG. 1 is a side view of different elements forming this prosthesis, before assembly;

FIG. 2 is a perspective view of the humeral rod which this prosthesis comprises;

FIG. 3 is an axial sectional view of various elements forming this prosthesis, after assembly;

FIG. 4 is a perspective view, from the distal side, of a glenoid element which this prosthesis comprises;

FIG. 5 is a perspective view, from the proximal side, of this glenoid element;

FIG. 6 is a side view of this glenoid element, and FIG. 7 is an end view of this glenoid element from the distal side.

### DESCRIPTION OF A PREFERRED EMBODIMENT

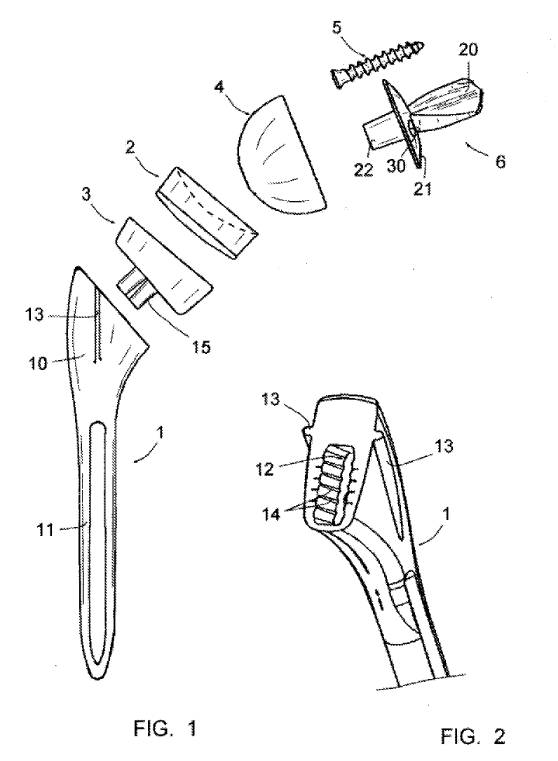

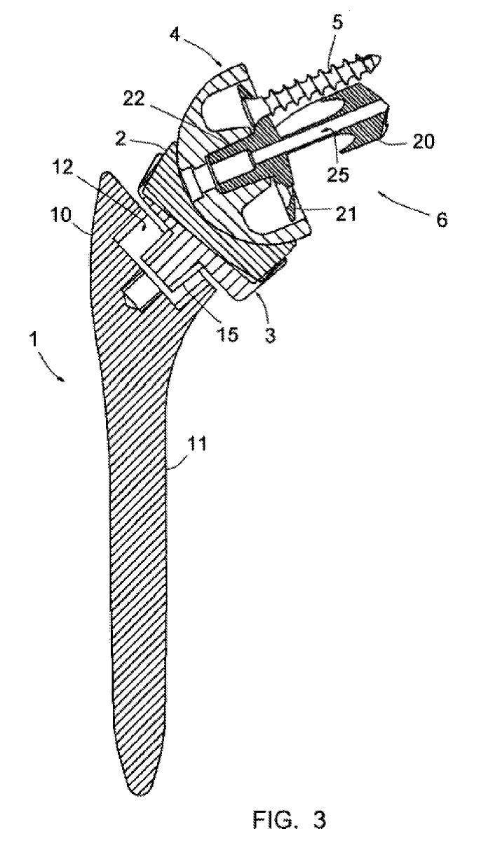

FIG. 1 illustrates various elements with which a shoulder prosthesis may be formed, i.e. a humeral medullary rod 1, an element 2 forming a glenoid jointed surface, a base 3 for mounting this element 2 on the humeral rod 1, a glenoid sphere 4 forming a convex rounded jointed surface able to cooperate with the jointed surface of the element 2, a bone anchoring screw 5 and a glenoid element 6.

The humeral rod 1 has a widened metaphysary portion 10 and a tapered epiphysary portion 11. The metaphysary portion 10 has a cavity 12 elongated in the internal-external plane, opening into a planar face which limits this metaphysary portion 10 on the proximal side, and comprises ribs 13 on its front and rear faces, ensuring its stabilization relatively to the bone. The humeral rod 1 may optionally receive a biological coating.

The cavity 12 comprises a plurality of protrusions 14 arranged at the front and rear walls which delimit it longitudinally; said base 3 comprises a stud 15 intended to be engaged into this cavity 12, the width of which is substantially smaller than the length of the cavity 12 and the side faces of which have a shape which is complementary to that of the protrusions 14. This cavity 12 and this stud 15 thus form an indexation system providing a plurality of possible positions of the base 3 with respect to the rod 1, and therefore an adjustment of the position of the element 2 with respect to this rod 1.

The element 2 is in a material promoting sliding, notably in high density polyethylene or in ceramic.

The base 3 in addition to the aforementioned stud comprises an adjusted housing for receiving the element 2.

The glenoid sphere 4 has a rounded, hemispherical shape or is like a spherical cap. It may be metal or may comprise a ceramic element, forming said jointed surface, this element being mounted on an insert in metal material.

The screw 5 is a bone screw of standard type.

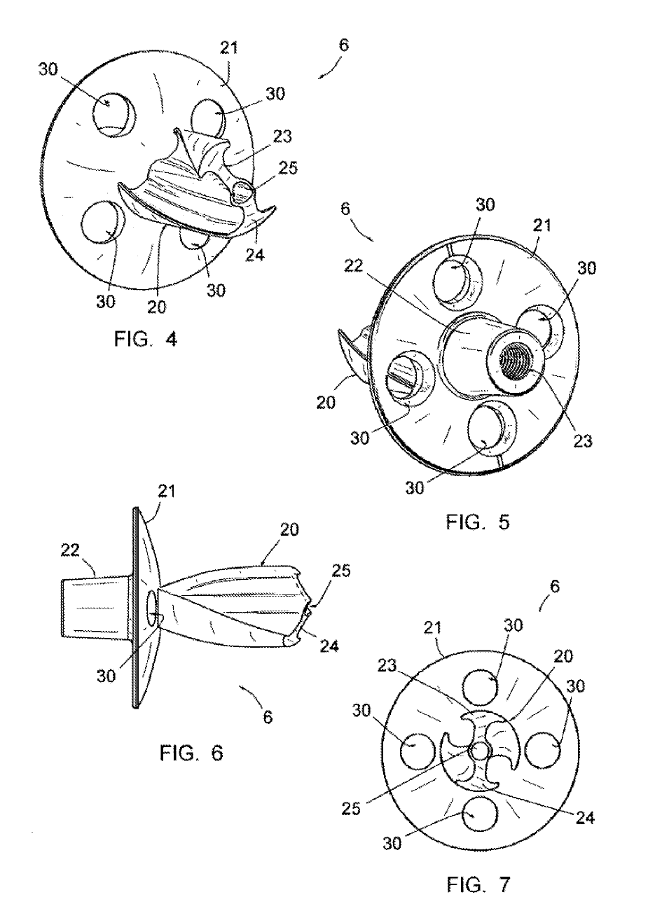

The glenoid element 6 is more particularly visible in FIGS. 4-7. As this appears with reference to these figures, this element 6 comprises a distal anchoring tail 20, a proximal flange 21 and a proximal stud 22 for mounting the glenoid sphere 4.

The distal anchoring tail 20 has a substantially “I-shaped” cross-section, i.e. with a central core and two side walls substantially perpendicular to this central core, and has a helicoidally twisted shape. Its distal end is arranged like the cutting end of a drilling bit, i.e. it has distal faces 23, 24 tilted on the proximal side from the axis of the distal anchoring tail (20) (cf. FIG. 6) and beveled according to inverse relief angles (cf. FIG. 4).

The distal anchoring tail 20 comprises an axial bore 25 allowing its engagement on a guiding pin (not shown).

Additionally, this distal anchoring tail 20 comprises an osteo-inductive coating, notably a calcium hydroxylapatite porous coating.

The proximal flange 21 has a distal face with a convex rounded shape, also comprising an osteo-inductive coating. It is perforated with four holes 30 regularly distributed on its periphery, which are each delimited by a wall as a hollow sphere portion, with which the screw 5 intended to be engaged through one of these holes 30 may be implanted according to a plurality of possible angular positions relatively to the glenoid element 6.

The pin 22 has a low-pitched frustro-conical shape and may receive the glenoid sphere 4 which is fitted on it, this head comprising a corresponding low-pitched frustro-conical cavity.

The pin 22 further comprises an axial tapped bore 23 for ensuring the mounting of the glenoid sphere 4 by means of a screw, if necessary, and optionally allowing extraction of the glenoid sphere by a system of the hub extractor type.

The glenoid element 6 is intended to be implanted by means of a set of instruments (not shown) including a localization instrument and a perforating instrument.

With the localization instrument, it is possible to localize the bone portion to be perforated; it has a flattened circular head in which an aperture is made with the same profile as the “I-shaped” cross-section of the medullary anchoring tail 20.

The perforating instrument is provided with a portion for perforating and hollowing out the bone of the socket of the scapula, with a same shape and same dimensions as those of the distal anchoring tail 20, or slightly smaller dimensions. The perforating instrument also comprises an axial bore allowing its engagement onto the same guiding pin as the one onto which the glenoid element 6 may be engaged.

In practice, the area to be perforated is localized by means of the localization instrument and the guiding pin is then set into place. The perforating instrument is then used for perforating and hollowing out the socket of the scapula according the shape of the distal anchoring tail of the element. This instrument is removed after perforation and the glenoid element 6 is then set into place by helicoidal impaction until the flange 21 comes into contact with the bone. One or more screws 5 are then placed through one of the holes 30. The glenoid sphere 4 is then engaged onto the pin 22 and, if necessary, secured in its mounting position by means of a screw set into place in the bore 23.

At the same time, the rod 1 is set into place in the humerus, and the element 2—base 3 assembly is set into place on it, in the adequate position by engaging the pin 15 into the cavity 12.

As this is apparent from the foregoing, the invention provides a shoulder prosthesis, the glenoid element of which has determining advantages of being perfectly attachable to the scapula, and this is relatively easy and rapid to apply.

It is obvious that the invention is not limited to the embodiment described above as an example but that it extends to all embodiments covered by the appended claims.

1-13. (canceled)

14. A shoulder prosthesis, comprising a glenoid element (6) intended to be attached to the scapula, wherein:
this glenoid element (6) comprises a distal anchoring tail (20) and a proximal flange (21), the distal anchoring tail (20) having a substantially “I-shaped” cross-section, i.e. with a central core and two side walls substantially perpendicular to this central core, and with a helicoidally twisted shape, and the proximal flange (21) being perforated with at least one hole (30); and
the prosthesis comprises at least one screw (5) intended to be engaged into the hole (30) of said flange (21) and inserted into the bone.

15. The shoulder prosthesis according to claim 14, wherein the distal helicoidal anchoring tail (20) comprises an osteo-inductive coating, notably a porous coating of calcium hydroxyapatite.

16. The shoulder prosthesis according to claim 14, wherein the distal end of the distal anchoring tail (20) is arranged in the same way as the cutting end of a drilling bit, i.e. it has distal faces (23, 24) tilted on the proximal side from the axis of the distal anchoring tail (20) and beveled according to inverse relief angles.

17. The shoulder prosthesis according to claim 14, wherein the distal anchoring tail (20) comprises an axial bore (25) allowing it to be engaged onto a guiding pin.

18. The shoulder prosthesis of claim 14, wherein the proximal flange (21) has a distal face with a convex rounded shape.

19. The shoulder prosthesis of claim 14, wherein the proximal flange (21) comprise an osteo-inductive coating

20. The shoulder prosthesis of claim 14, wherein at least one of the holes (30) of the flange (21) may be delimited by a conical wall or a hollow sphere portion, allowing the screw (5) to be implanted according to a plurality of possible angular positions relatively to the glenoid element (6).

21. The shoulder prosthesis of claim 14, wherein the flange (21) comprises several holes (30) notably four holes.

22. The shoulder prosthesis of claim 14, wherein the glenoid element (6) comprises mounting means on it for an element forming the jointed surface, including notably a low-pitched frustro-conical stud (22), onto which an element (4) forming the jointed surface may be engaged, this element (4) comprising a corresponding low-pitched frustro-conical cavity.

23. The shoulder prosthesis of claim 22, wherein the stud (22) comprises an axial tapped bore (23) with which the mounting of the element (4) forming the jointed surface may be ensured by means of a screw.

24. A set of instruments for implanting the prosthesis of claim 22, wherein it comprises a perforating instrument, provided with a portion for perforating and hollowing out the bone of the socket of the scapula having a same shape and same dimensions as those of the distal anchoring tail (20) of the glenoid element (6), or slightly smaller dimensions.

25. The set of instruments of claim 24, wherein the perforating instrument comprises an axial bore allowing it to be engaged onto a guiding pin.

26. The set of instruments of claim 24, wherein it comprises an instrument for locating the bone portion to be perforated, having a flattened circular head in which an aperture is made of the same profile as that of the “I-shaped” cross-section of the medullary anchoring tail.

27. A set of instruments for implanting the prosthesis of claim 14, wherein it comprises a perforating instrument, provided with a portion for perforating and hollowing out the bone of the socket of the scapula having a same shape and same dimensions as those of the distal anchoring tail (20) of the glenoid element (6), or slightly smaller dimensions.

28. A set of instruments for implanting the prosthesis of claim 15, wherein it comprises a perforating instrument, provided with a portion for perforating and hollowing out the bone of the socket of the scapula having a same shape and same dimensions as those of the distal anchoring tail (20) of the glenoid element (6), or slightly smaller dimensions.

29. A set of instruments for implanting the prosthesis of claim 16, wherein it comprises a perforating instrument, provided with a portion for perforating and hollowing out the bone of the socket of the scapula having a same shape and same dimensions as those of the distal anchoring tail (20) of the glenoid element (6), or slightly smaller dimensions.

30. A set of instruments for implanting the prosthesis of claim 17, wherein it comprises a perforating instrument, provided with a portion for perforating and hollowing out the bone of the socket of the scapula having a same shape and same dimensions as those of the distal anchoring tail (20) of the glenoid element (6), or slightly smaller dimensions.

31. A set of instruments for implanting the prosthesis of claim 18, wherein it comprises a perforating instrument, provided with a portion for perforating and hollowing out the bone of the socket of the scapula having a same shape and same dimensions as those of the distal anchoring tail (20) of the glenoid element (6), or slightly smaller dimensions.

32. A set of instruments for implanting the prosthesis of claim 19, wherein it comprises a perforating instrument, provided with a portion for perforating and hollowing out the bone of the socket of the scapula having a same shape and same dimensions as those of the distal anchoring tail (20) of the glenoid element (6), or slightly smaller dimensions.

33. A set of instruments for implanting the prosthesis of claim 20, wherein it comprises a perforating instrument, provided with a portion for perforating and hollowing out the bone of the socket of the scapula having a same shape and same dimensions as those of the distal anchoring tail (20) of the glenoid element (6), or slightly smaller dimensions.
